(19) United States
(12) Patent Application Publication
(10) Pub. No.: US 2008/0245233 A1
(43) Pub. Date: Oct. 9, 2008

(54) Method for Inhibiting Fouling in Basic Washing Systems

(75) Inventors: Zhenning Gu, Sugar Land, TX (US); Joseph L. Stark, Richmond, TX (US); Roger D. Metzler, Sugar Land, TX (US)

Correspondence Address: MADAN, MOSSMAN & SRIRAM, P.C., 2603 AUGUSTA DRIVE, SUITE 700, HOUSTON, TX, 77057-5662, US

(73) Assignee: BAKER HUGHES INCORPORATED, Houston, TX (US)

(21) Appl. No.: 12/062,359

(22) Filed: Apr. 3, 2008

Related U.S. Application Data
(60) Provisional application No. 60/910,413, filed on Apr. 5, 2007.

(51) Int. Cl. B01D 53/00; C01B 17/64; C01B 17/98; C09K 3/00; C07C 243/12; C07C 243/22; C07C 239/08; C07D 263/02

(52) U.S. Cl. 95/149; 423/514; 423/519.2; 252/384; 252/385; 564/464; 564/310; 564/301; 548/215

(57) ABSTRACT

Fouling in basic washing systems such as caustic scrubbers can be prevented or at least mitigated by treating the liquid washing phase used in a caustic scrubber with an additive having at least one compound selected from the group consisting of oxalyl dihydrazide, a disulfite salt, isopropyl hydroxylamine, chlorobenzhydrazide, aminobenzhydrazide, a thiosulfate salt, 4,4-dimethyl oxazolidine, a hydrosulfite salt, and mixtures thereof.

### CROSS-REFERENCES TO RELATED APPLICATIONS

This application claims priority from U.S. Provisional Patent Application Ser. No. 60/910,413, filed Apr. 5, 2007.

### BACKGROUND OF THE INVENTION

1. Field of the Invention

The present invention relates to a method for inhibiting fouling in basic washing systems. The present invention particularly relates to the use of additives for inhibiting fouling in basic washing systems.

2. Background of the Art

Gas Scrubbers are devices used for separating components of a gas admixture. In some embodiments, these devices are used to “purify” gasses or, stated in the alternative, remove undesirable components from a gas stream. For example, primitive scrubbers have been used since the inception of submarine warfare to remove carbon dioxide from the air supply in the submarine.

More recently, gas scrubbers have proven to be essential in many industries. For example, Gas Scrubbers are used to prevent pollution from the burning of coal during power generation. Gas Scrubber are also used to remove undesired components from crude oil during refining and to remove undesirable components from process gas streams during the production of chemicals, metals and devices such as semiconductors and the like.

### SUMMARY OF THE INVENTION

In one aspect, the invention is a method for the prevention or mitigation of fouling in basic washing systems comprising treating a liquid washing phase used in a basic washing system with an additive comprising at least one compound selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

In another aspect, the invention is a composition useful as an additive for preventing or mitigating the formation of fouling within a basic washing system comprising at least one compound selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

In yet another aspect, the invention is a method for mitigating the fouling of a caustic scrubber used to wash a gas steam including ethylene comprising treating a liquid washing phase used in the caustic scrubber with an additive comprising a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

### DESCRIPTION OF THE PREFERRED EMBODIMENTS

One embodiment of the invention is a method for the prevention or mitigation of fouling in a basic washing system. The most common basic washing systems are caustic scrubbers. For the purposes of the present application, a caustic scrubber is device for removing water soluble and/or acidic or other base reactive components from a fluid stream, often a gas. Also for the purposes of the present application, the term “caustic” is defined broadly to mean a strong base (alkaline) substance including, but not limited to sodium hydroxide, potassium hydroxide, and lithium hydroxide; but also specifically including any compound now known or after discovered useful for extracting a water soluble and/or acidic or other base reactive component from a fluid stream in a fluid scrubber. For example, the basic washing systems useful with the disclosure may include an organic amine or a solution including an organic amine as a liquid scrubbing material.

Conventional gas scrubbers include a “tower” which is, in essence, a pipe or column, typically including trays or occasionally an inert packing, where a gas stream is contacted with a liquid scrubbing material. In the case of at least one embodiment of the invention, the liquid scrubbing material is a caustic liquid as defined herein. In some applications, the liquid scrubbing material is passed down through an upward moving gas stream with a packing serving to increase the mixing of the gas and liquid scrubbing material. In an alternative embodiment, the direction of the gas and fluid passing through the tower may be reversed from the first embodiment. In still another embodiment, the gas stream and the caustic fluid of the method of the application may move in the same direction.

There are many types of gas scrubbers which are included within the meaning of the term caustic scrubber that are known to be useful. For example, U.S. Pat. No. 6,284,019 to Sorensen, et al., which is fully incorporated herein by reference, discloses a scrubber for removing pollutants from a gas comprising a housing for conducting the gas therethrough, a mechanism for introducing pollutant-reactive filtering agent into the housing, a sump for collecting the filtering agent and having a first volume, and an intermediate sump for collecting the filtering agent and channeling the filtering agent into the sump, the intermediate sump having a second volume that is less than the first volume. Rather than having a tower, this patent discloses the use of a “chamber” which serves the same function as a tower and, for the purposes of the invention shall be designated as such.

A variety of scrubbers are commercially available. For example, the TRI-MER Corporation markets a variety of scrubbers for use in industry. Exemplary are the so called “cross flow” scrubbers, which come in a variety of configurations. Common to most commercial scrubbers is a “sump” and/or storage tank for scrubbing liquids. These sumps and/or storage tanks may be internal or external to the scrubber cabinets. Any gas scrubber which utilizes a caustic liquid in order to scrub a gas and is known to be useful to those of ordinary skill in the art of using gas scrubbers may be used with embodiments of the method of the disclosure.

With caustic scrubbers, the caustic liquid in the scrubber is sometimes consumed with use, especially when the liquid scrubbing material is an inorganic base such as sodium hydroxide. As a consequence, in such embodiments, the liquid scrubbing material may be renewed either continuously or intermittently. Ideally, it would be desirable in the art that the caustic liquid scrubbing material be renewed only as it is consumed, that is as a consequence of the loss of alkalinity due to reaction of the caustic liquid scrubbing material with acidic, or base reactive, compounds in the fluid being scrubbed. Unfortunately, the scrubber may require a more frequent renewal of the caustic fluid, if not an actual shut down and clean out, due to the phenomena of fouling.

While caustic scrubbers are a very common embodiment of basic washing systems, there are other types of basic washing systems in use. Other examples of basic washing systems include scrubbers that use alkanolamines, (such as methyl ethylamine (MEA), diethyl amine (DEA), methyl diethylamine (MDEA), and amine diisopropanol (ADIP)), hindered amines, and alkazide as the liquid washing materials. Still other types of basic washing systems include water wash columns such as those used to refine crude butadiene in butadiene manufacturing plants. Some washing systems are liquid/liquid washing systems where both the washing materials and the stream being washed are liquids and at least some embodiments of the invention may be used with these applications as well.

For the purposes of the application, fouling, in relation to a basic washing system, occurs when chemical reactions occur in the caustic liquid scrubbing material resulting in solids (or sludges) and/or substantial increases in the viscosity of the caustic liquid scrubbing material.

While not wishing to be bound by any theory, it is never-the-less believed that when fluids including unsaturated compounds are washed; at least some fouling is the result of aldol polymerizations. In an aldol polymerization, often referred to in the art as an aldol condensation, two molecules, each one having an aldehyde or ketone group react to form a single molecule having a hydroxyl and a carbonyl group. Embodiments of the method of the invention are particularly useful for scrubbing gas streams having components that may undergo an aldol condensation. For example, in one embodiment, the method of the disclosure is used to wash gas including ethylene using a caustic scrubber.

Refineries and Chemical Plants are among those industries most likely to have fluid streams and especially gas streams which include either acidic or base reacting gases, or both, that are undesirable and also include aldehyde and/or ketones. Embodiments of the method of the application may be useful in applications where, except for causing fouling, the aldehyde and/or ketone component of a fluid stream is not otherwise undesirable. Embodiments of the method of the invention are also useful in applications where it is desirable to remove an aldehyde or ketone component of a gas stream.

In one embodiment of the invention, it is noted that failure to scrub or adequately scrub a gas stream that has a tendency to foul scrubbers is often undesirable in industry. Fouling in scrubbers can be the cause of increased maintenance costs and lost productivity where products are either made a slower rate because of poor scrubber efficiency or products produced are out of specification. As an example of the latter, consider a gas stream which may have a CO₂ and/or H₂S specification. Failure to meet the specification may require that the product gas be sent through a second scrubber or refused by a consumer. Either of these can cause production costs to rise with a consequential loss of profits.

Scrubber failures due to fouling may also have safety and environmental consequences. While fouling may be most noticeable in the tower of a scrubber, it may also occur in the sump, holding tank, and in any other pipe, vessel, or other portion of a scrubber where caustic liquid scrubbing material has sufficient residence time to allow for the dropping of suspended solids or the forming of a film or coating onto the walls of the exposed portions of the scrubber.

In some embodiments of the method of the disclosure, a scrubber is treated with an additive. The additive includes at least one member of the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof. For the purposes of this application, salts of these compounds that may form salts are included within the scope of the disclosure even if not specifically specified. For example, even though isopropyl hydroxylamine hydrochloride is not specifically listed, an application where this amine hydrochloride was added to a caustic solution, and thus converted back to an amine, would be within the scope of the disclosure and the claims of this application.

The cations for the salts listed therein may be any cation which would result in a caustic solution soluble salt. For Example, sodium thiosulfate and potassium thiosulfate may be used as additives with at least some embodiments of the method of the application. In one embodiment of the invention, the additives includes at least one member of the group consisting of: oxalyl dihydrazide, potassium disulfite, isopropyl hydroxylamine (IPHA), 2-chlorobenzhydrazide, 4-aminobenzhydrazide, sodium thiosulfate, 4,4-dimethyl oxazolidine, sodium hydrosulfite, and mixtures thereof. While, in one embodiment, the additive is a solid which can be added directly or indirectly to a caustic liquid scrubbing material, in other embodiments, the additive is added as a solution or suspension.

The additives of the present invention may be in the form of a aqueous solution, possibly including a dispersant, of oxalyl dihydrazide, chlorobenzhydrazide, aminobenzhydrazide, potassium disulfite, isopropyl hydroxylamine (IPHA), sodium thiosulfate, 4,4-dimethyl oxazolidine, sodium hydrosulfite, and mixtures thereof. When the additives are in the form of a solution, generally the concentration the oxalyl dihydrazide, chlorobenzhydrazide, aminobenzhydrazide, potassium disulfite, isopropyl hydroxylamine (IPHA), sodium thiosulfate, 4,4-dimethyl oxazolidine, sodium hydrosulfite, and mixtures thereof will be from about 0.1 to about 50 weight percent. In some embodiments, the concentration will be from about 5 to about 30 weight percent and in others, from about 7 to about 20 weight percent.

Any dispersant useful for dissolving or suspending one or more of these compounds in water may be used with at least some of the embodiments of the method of the application. In addition, one of the compounds may be dissolved or suspended in a water soluble or miscible solvent. Exemplary of such materials are: mono-ethylene glycol n-hexyl ether (Hexyl Cellosolve® available from Union Carbide); ethylene glycol monobutyl ether (Butyl Cellosolve®); di- and tri-propylene glycol derivatives of propyl and butyl alcohol, which are available from Arco Chemical (3801 West Chester Pike, Newtown Square, Pa. 19073) and Dow Chemical (1691 N. Swede Road, Midland, Mich.) under the trade names Arcosolv® and Dowanol®; mono-propylene glycol mono-propyl ether; di-propylene glycol mono-propyl ether; mono-propylene glycol mono-butyl ether, di-propylene glycol mono-propyl ether, di-propylene glycol mono-butyl ether; tri-propylene glycol mono-butyl ether; ethylene glycol mono-butyl ether; di-ethylene glycol mono-butyl ether, ethylene glycol mono-hexyl ether; di-ethylene glycol mono-hexyl ether; 3-methoxy-3-methyl-butanol; and mixtures thereof. In regard to these solvents, “butyl” includes normal butyl, isobutyl and tertiary butyl groups. Mono-propylene glycol and mono-propylene glycol mono-butyl ether can be used and are available under the tradenames Dowanol DPnP® and Dowanol DPnB®. Di-propylene glycol mono-t-butyl ether is commercially available from Arco Chemical under the tradename Arcosolv PTB®. In some instances, it might be preferred to use combinations of these solvents, such as Hexyl cellusolve with Butyl cellusolve, or Dowanol PnB with 3-methoxy-3-methyl-butanol.

The additives of the application are desirably fed to basic washing systems such as scrubbers at an effective concentration. Those of ordinary skill in the art of running such units are well versed in determining the effective concentration of additives to use in their equipment. Such concentrations are dictated, in the case of gas scrubbers for example, by the operational conditions of the scrubbers including the makeup of the gas stream, feed rates, and operating temperatures. Generally, the additives will be present at a level in the caustic solutions in the scrubbers such that the concentration of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 100 to 50,000 ppm. In other embodiments the concentration is from 1000 to 5000 ppm.

The additives may be desirably added to a liquid feed stream into a basic washing system such as, for example, a caustic scrubber. The additive may be added directly to the caustic scrubber or aspirated into a gas feed stream. The additives maybe also be introduced into a basic washing system using any other method known to be useful for introducing an additive to a scrubber.

In addition to the additive components already described, the additives of the invention may include other compounds known to be useful in basic washing systems such as dispersants, defoamers, and the like. Any compound that does not have an undesirable interaction with the additive's ability to prevent fouling may be used with at least some embodiment of the method of the invention.

### EXAMPLES

The following examples are provided to illustrate the present invention. The examples are not intended to limit the scope of the present invention and they should not be so interpreted. Amounts are in weight parts or weight percentages unless otherwise indicated.

### Example 1

A blank was prepared by admixing spent caustic bottoms (pre-filtered to remove existent solid particles) dosed with 6000 ppm of vinyl acetate and with 5% oil. Samples 1-1 through 1-6 are prepared by admixing the blank material with the additive shown below in Table 1. The dosed caustic sample was heated at 80° C. for sixteen hours. Once the heating period was complete the sample was allowed to cool to ambient. The caustic was filtered through a 1.0-1.5 μm-7.0 cm glass fiber filter. The filter paper was dried and weighed. The weight gain is reported as potential polymer/fouling formation. Inhibitor efficacy is measured in comparison to sample blank.

TABLE 1

| | | Additive | | |
|---|---|---|---|---|
| Sample | | Dosage | Potential Sediment | % Fouling |
| # | Additive dosed | (ppm) | (mg/100 ml) | Inhibition |
| BLANK | — | — | 245.5 to 315.0** | — |
| 1-1 | Oxalyl | 8200 | 89.0 | 66.9 |
| | Dihydrazide | | | |
| 1-2 | Potassium | 4650 | 112.7 | 64.2 |
| | Disulfite | | | |
| 1-3 | IPHA | 5250 | 116.7 | 52.5 |
| 1-4* | NaBH₄ | 660 | 208.1 | 33.6 |
| 1-5 | Sodium | 11000 | 235.5 | 25.1 |
| | Thiosulfate | | | |
| 1-6 | 4,4-Dimethyl | 9400 | 241.8 | 10.2 |
| | Oxazolidine | | | |

*Comparative Example.
**The blank material appeared to degrade with time. Blanks were rerun with each test and % fouling was determined using a blank run near in time with the designated sample.

### Example 2

10 ml of 2% NaOH was added to 20 ml glass vials. One vial is used as a blank and left untreated. The remaining vials are treated with sufficient vinyl acetate to result in a concentration of 1100 ppm. One of the vials is sealed used as a control. The remaining vials are treated with additives as shown below in Table 2. All four vials were capped and shaken well before settling in an oven at 55° C. overnight. Table 2 shows the results of observation.

TABLE 2

| Sample | Inhibitor | Result |
|---|---|---|
| Blank | None | Clear, colorless |
| Control | None | Hazy, yellow colored |
| 2-1 | 2-chlorobenzoic | Clear, very light yellow colored |
| | hydrazide | |
| | (3400 ppm) | |
| 2-2 | 4-aminobenzoic | Clear, ivory colored |
| | hydrazide | |
| | (3400 ppm) | |

### Example 3

A control sample was prepared by dosing 6000 ppm of vinyl acetate into 2% NaOH solution. Samples are prepared by dosing into a control sample the additives shown below in Table 3. The dosed caustic sample was heated at 80° C. for sixteen hours. Once the heating period was complete, the sample was allowed to cool to ambient. The caustic was filtered through a 1.0-1.5 micrometer glass fiber filter that was 7.0 cm in diameter. The filter paper was dried and weighed. The weight gain is reported as potential polymer/fouling formation. Inhibitor efficacy is measured in comparison to the control sample.

TABLE 3

| | | Additive | Potential | |
|---|---|---|---|---|
| Sample | | Dosage | Sediment | % Fouling |
| # | Additive dosed | (ppm) | (mg/100 ml) | Inhibition |
| Control | — | — | 112.0-118.4 | — |
| 3-1 | 25% Sodium | 3000 | 54.1 | 54.3 |
| | hydrosulfite in | | | |
| | Caustic Solution | | | |
| 3-2 | 18% Sodium | 3750 | 25.4 | 78.5 |
| | hydrosulfite in | | | |
| | Caustic Solution | | | |

What is claimed is:

1. A method for the prevention or mitigation of fouling in basic washing systems comprising treating a liquid washing phase used in the basic washing system with an additive comprising a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

2. The method of claim 1 wherein the basic washing system is a scrubber.

3. The method of claim 2 wherein the scrubber is a gas scrubber.

4. The method of claim 3 wherein the gas scrubber is used to wash a gas including ethylene.

5. The method of claim 1 wherein the basic washing system is used to wash a fluid including unsaturated compounds.

6. The method of claim 5 wherein the unsaturated compounds are those that may undergo an aldol condensation.

7. The method of claim 1 wherein the liquid washing phase is a caustic liquid.

8. The method of claim 7, wherein the caustic liquid comprises an aqueous solution of a member selected from the group consisting of sodium hydroxide, potassium hydroxide, lithium hydroxide and mixtures thereof.

9. The method of claim 8 wherein the caustic liquid comprises an aqueous solution of sodium hydroxide.

10. The method of claim 1 wherein the additive includes a compound selected from the group consisting of potassium disulfite, sodium thiosulfate, sodium hydrosulfite, and mixtures thereof.

11. The method of claim 1 wherein the additive comprises an aqueous solution and wherein the concentration of the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 0.1 to about 50 weight percent.

12. The method of claim 1 wherein the additive comprises an aqueous solution and wherein the concentration of the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 5 to about 30 weight percent.

13. The method of claim 1 wherein the additive comprises an aqueous solution and wherein the concentration of the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 7 to about 20 weight percent.

14. The method of claim 1 wherein the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof in the liquid washing phase is present at a concentration of from about 100 to about 50,000 ppm.

15. The method of claim 1 wherein the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof in the liquid washing phase is present at a concentration of from about 1,000 to about 5,000 ppm.

16. A method for mitigating the fouling of a caustic scrubber used to wash a gas including ethylene comprising treating a liquid washing phase used in the caustic scrubber with an additive comprising a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

17. The method of claim 16 wherein the disulfite salt is potassium disulfite, the thiosulfate salt is sodium thiosulfate, and the hydrosulfite salt is sodium hydrosulfite

18. A composition useful as an additive for preventing or mitigating the formation of fouling within a basic washing system comprising at least one compound selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

19. The composition of claim 18 wherein the composition includes at least one member of the group consisting of potassium disulfite, sodium thiosulfate, sodium hydrosulfite, and mixtures thereof.

20. The composition of claim 19 further comprising a dispersant and/or a defoamer.
